(19) United States
(12) Patent Application Publication
(10) Pub. No.: US 2014/0000624 A1
(43) Pub. Date: Jan. 2, 2014

(54) ADAPTABLE LARYNGEAL MASK

(71) Applicant: Donald Munro Miller, Cape Town (ZA)

(72) Inventor: Donald Munro Miller, Cape Town (ZA)

(21) Appl. No.: 14/005,965

(22) PCT Filed: Mar. 22, 2012
(86) PCT No.: PCT/IB12/51366

§ 371 Date: Sep. 18, 2013

(30) Foreign Application Priority Data
Mar. 23, 2011 (GB) 1104865.9

(51) Int. Cl. A61M 16/04

(52) U.S. Cl. CPC A61M 16/0434
USPC 128/207.15

(57) ABSTRACT

An airway device (10) is provided which includes a distal part (12) and a proximal part (20) in sealed communication. The distal part (12) has a distal opening (14) for attachment to a breathing apparatus, in sealed communication with a hollow stem (16). The proximal part comprises a hollow sealing chamber (20) with a proximal opening (22), said sealing chamber (20) having means for sealing within and against the mucosal walls of a body cavity with a corresponding proximal opening, for communicating with the lungs. The sealing means of the sealing chamber (20) includes: a longitudinal fold (32) that allows for adaptation to varying cross-sectional dimensions of said body cavity; and said sealing chamber (20) being made of an elastomeric substance and having walls (26,30) that are thicker and relatively less flexible, and walls (24,25) that are thinner and relatively more flexible, said thicker walls (26,30) forming a framework that supports said thinner walls (24,25).

### FIELD OF THE INVENTION

The present invention relates to respiratory apparatus in the form of an artificial airway device for placement into the oro-pharynx or trachea of an unconscious patient.

### BACKGROUND TO THE INVENTION

In order to support respiration and therefore life, an unconscious patient may require some or all of the following objectives, namely: the maintenance of airway patency, attachment to respiratory apparatus, either spontaneous or controlled positive pressure ventilation, prevention of inhalation into the lungs of extraneous matter such as vomitus or blood.

During long term ventilation in intensive care, seepage of liquids from above the sealing cuffs of tracheal tubes may cause a problem of nosocomial infection of the lungs and a potential problem from pressure damage with sealing tracheal cuffs, both mucosal and recurrent laryngeal nerve damage. Pressure damage from the sealing cuffs of supraglottic airways may also result in mucosal trauma and, more seriously, nerve damage to nerves that could affect speech, such as recurrent laryngeal and hypoglossal nerves. In addition, the blind placement of a tracheal tube into the trachea and blind placement of a gastric tube into the oesophagus may be required.

The words “proximal” (near the patient) and “distal” (further from the patient) as used herein, provide references with respect to the patient.

During anaesthesia or resuscitation, airway management may be achieved by means of an endotracheal tube with an inflatable cuff around the proximal end of the tube, which is placed with the help of other instruments within the trachea. The disadvantage is the need for accurate placement that requires specially trained skills and it is more invasive requiring the placement of a tube into a relatively sterile area and may also require a laryngoscope for placement. Its placement often requires the administration of muscle paralyzing drugs. Gastric tubes may be passed into the stomach for the purpose of removing possible fluid contents via the oral or nasal routes after the airway has been secured using a tracheal tube. Once tracheal tubes have been placed, the cuff pressures may rise excessively causing damage to the mucosa—or rarely even to underlying nerves or nerve endings.

Inflatable cuffs in tracheal tubes themselves can cause pressure damage where the recurrent laryngeal nerve lies in close proximity to the trachea. More commonly, bacteria-containing secretions descending from the pharynx, can seep down past the folds in the cuff of cuffed tracheal tubes, producing infection in the lungs.

Many of these problems could be overcome by using a type of laryngeal mask airway (LMA™). This may involve the use of an inflatable cuff surrounding a bowl shaped end of a tube for sealing the entrance to the larynx. It is less invasive than endotracheal tubes described above and it does not require muscle relaxation. However, regurgitation of liquid vomitus or blood can very easily get into the bowl of the LMA and lead to pulmonary aspiration. In addition, the perilaryngeal seal is not of a very high quality and there may be a limitation to the inflation pressure that can be used. When higher inflation pressures are used, air may be forced down the oesophagus and into the stomach—producing gastric distension or regurgitation of liquids that are within the stomach.

Improvements to both these limiting factors are found in slightly more complicated further developments of supraglottic airway devices. The oesophageal obturator airway (EOA) named “Combitube®” or derivative (Suction Laryngeal Tube) or modification of the laryngeal mask called the ProSeal LMA, provide drainage tubes that will allow air and liquids that get into the oesophagus, to escape from the oesophagus. Devices containing drainage tubes often permit the passage of an oro-gastric tube into the stomach as may be required for some surgical procedures.

Each of the Combitube and Suction Laryngeal tube comprises a double lumen, double cuffed tube, the longer tube with attached cuff passes into the oesophagus for the purpose of sealing and isolating contents which may enter the oesophagus from below or to prevent the escape of gas under pressure from above from entering the stomach. The shorter tube for ventilating the lungs ends within the pharynx, the oro-nasal outlet from the pharynx being sealed off within the pharynx by means of the second cuff which surrounds both tubes, which when inflated, allows for positive pressure to develop within the pharynx.

The cuffed oro-pharyngeal airway (COPA®) has been introduced by Mallinckrodt Medical, Inc, (U.S. Pat. No. 5,743,256) and also the Cobra airway, which can be used to achieve some of the objectives stated above but both fail to protect the lungs from extraneous matter that enters the pharynx, from entering the lungs. The COPA is no longer in production. The glottic aperture seal airway of Augustine Medical Inc. (WO 98/16273), purports to achieve this advantage, however, it is not as reliable as was originally hoped. Numerous other double cuff inflating devices are appearing in the market and may be classed as derivatives of the EOA and COPA above, e.g. that of Sato et al (U.S. Pat. No. 5,743,258).

The most recent supraglottic airways that seal in the pharynx are those without any inflatable cuffs and they include the SLIPA™ airway, the i-gel airway (WO 2004/016308) and the Baska Airway device (U.S. 2006/0180156). The SLIPA airway was designed by the inventor in the present application, was the first of these devices and comprises a tube supplying a hollow anatomically shaped plastic chamber that lines the pharynx. The chamber has an orifice which corresponds with the patient's larynx. The chamber acts as a trap for liquids from any source and so has a pulmonary aspiration protective role. The device seals at the base of the tongue in common with the Combitube, Laryngeal tube, COPA and Cobra airways. The sealing mechanism of the SLIPA is an anatomically pre-shaped chamber that lines the pharynx. The seal relies upon the resilience of the blow moulded material that is stiff enough to be held against the walls of the pharynx and this is further supported by the airway pressure on the inside of the wall of the chamber. (Anesthesia analgesia 2004, vol 99).

It should be noted that the prototype silicone versions of the SLIPA airway in 1995 were made of thin walled silicone material, where the airway pressure maintained an excellent quality self-energizing seal by virtue of the thin walls of the device being held firmly against the mucosal walls by the airway pressure. It did not go into production at that time or become available to the public.

The i-gel is a peri-laryngeal sealing device like the LMA group, but instead of using an air cushion, it uses a very flexible soft solid material cuff using materials of low Shore hardness to achieve a pharyngeal seal. The aspiration protection mechanism employed here is very similar to the drainage tube employed in the ProSeal laryngeal mask airway device.

The maximum inflation pressure that can be used before gas leakage occurs around the solid or air inflated cuffs, is limited by the force of wedging of a solid cuff or the maximum permissible inflation pressure of the cuff that will prevent tissue damage. Should regurgitation occur, due to the substantial volumes occupied by air inflated or solid soft material sealing cuffs, there is limited storage of regurgitated material or other secretions or blood. Laryngeal masks and i-gel provide a partial seal and isolation of the oesophagus from airway pressure and possibly regurgitation. However, liquids can seep past the weak airway sealing components and regurgitated material can pass into the ‘bowl’, which comprises part of the airway channel. If this happens, due to the limited storage capacity resulting from high volume sealing mechanisms, even small volumes of liquids/material that pass into the bowl, are likely to pass into the lungs during inspiration.

The COPA and the more recent Cobra airways do not provide any seal of the oesophagus and there have been reported incidents of aspiration. Aspiration has even been reported in the presence of devices with drainage tubes.

The Combitube and its derivatives e.g. laryngeal tube series seal off the oesophagus and would appear to be effective for controlled ventilation, but its correct placement can pose problems—either too deep or not deep enough. Also, it has rather an elaborate action and is expensive.

To overcome the aspiration risk factors associated with the LMA, an improved LMA named the “ProSeal” LMA (and a more recent disposable version the LMA “Supreme”) incorporates a moderate bore oesophageal tube for removing liquid that may accumulate in the mask region of the airway by suction or siphonage (JP 2-283378).

More recently, the Baska mask airway employs a device to improve the limitation of regurgitation risk and aspiration by the design of a laryngeal mask with a cavity or sump at the leading end of the mask for collecting regurgitated liquids and for their effective rapid removal by means of two tubes—one for the application of suction and one to allow for air to be drawn into the sump where fluids are collected. This more effective active removal of regurgitation liquids may be more effective than devices with simple gastric tubes. The Baska airway (WO 2009/026628) includes a bellows action, self-energizing sealing mechanism.

The placement in the trachea of the endotracheal tube is the most effective means of achieving all of the above objectives, however, its use requires experience, skill and the use of a laryngoscope or specially designed supraglottic airway such as the intubating laryngeal mask (Fastrach), which in turn has its own unwanted side effects consequent upon powerful neural reflex actions. Its placement may also require the use of muscle paralyzing drugs.

All the above devices are made of soft, flexible materials and the need for a removably secure connection to respiratory apparatus necessitates a connecting mechanism with sufficient tenacity to prevent unintentional disconnection. The force required for detachment of the respiratory apparatus from the airway device should exceed 30 Newtons. For this to be achieved usually requires a hard plastic male connector that is applied (via barbed fitting or glued or stretched with good quality friction fit) to the softer material of the airway device. On rare occasions this junction has come adrift and constitutes a potential hazard, which would be beneficial to avoid, if possible. Furthermore, there are additional expenses involved in manufacture both in capital (additional moulds) and labour costs. Many functional and manufacturing advantages may be achieved if the part of an airway device that attaches to breathing apparatus, is made of the same softer materials as the rest of the airway device.

A recent development by the inventor in the present application, relates to a combined obturator and airway device named the SLIPA™ (also mentioned above) (WO 02/32490). This device addresses many of the objectives named above for supporting an unconscious patient, and like all current devices, it uses a standard 15 mm attachment connector that conforms to BS 5356 standard for conical connectors. These attachments are made of different materials from the airway devices, with the result that there can occasionally be insufficient frictional grip between the connectors and the devices and the connectors can therefore potentially come adrift—with potential risks. In addition, these connectors restrict the diameter of instruments that may pass through them. The use of larger instruments such as the passage of standard size of tracheal tubes has been a desirable objective for many years.

One development that is specifically intended for the passage of larger tubes is the AirQ laryngeal mask airway designed by Dr Daniel Cook, which is a laryngeal mask airway designed to have the 15 mm connector easily removable and re-attachable to allow the passage of larger tracheal tubes. Despite the need for inserting larger instruments through the airway, no supraglottic airway with any other attachment site than the 15 mm hard plastic or metal connector, has been used for airway devices.

Another potential complication of using supraglottic airways and tracheal tubes, is the incidence of damage to certain nerves, namely the hypoglossal and recurrent laryngeal nerves, due to pressure effects of cuff inflating airway devices in the pharynx. More recently, pressure injuries on the tip of the tongue from large bite-blocks have been noted. The SLIPA device, unlike many other airway devices, is pre-shaped with an indentation in the lateral aspect of the chamber just below the sealing ridge, which may be partially effective in preventing nerve injury at a particular site. However, its sealing site is at the base of the tongue and not around the larynx. All supraglottic sealing devices that seal in the immediate perilaryngeal area thus far described (namely laryngeal masks and their variants, including the i-gel airway), in contrast to the base-of-the-tongue-sealing devices, run the risk of applying pressure over the tip of the hyoid bone, which is in very close proximity to the hypoglossal nerve where one important type of nerve injury has been reported on a few occasions. That is because the perilaryngeal sealing site of necessity overlies the vulnerable area. The other reason there is potential injury at this site is that perilaryngeal sealers, as opposed to base-of-tongue-sealers, are concave towards the front with a sealing site that overlies the vulnerable area. The i-gel for instance is described as a device that “mirrors the laryngeal inlet”. To mirror is to achieve a seal by means of apposition of a matching structure (protuberance matched with protuberance and indentation matched with indentation) and may be contrasted with a possibly more desirable complementary arrangement that will be more compliant/complaisant with the surrounding tissues.

One object of the present invention is to provide an appropriately shaped artificial airway which will achieve a dynamic self-energizing seal in the peri-laryngeal region, as opposed to a constant pressure seal that is achieved through sealing cushions. During positive pressure ventilation, there is a change in the airway pressure throughout the respiratory cycle. This means that there is variation in the dimensions of the pharynx during each respiratory cycle with enlargement of the dimensions as the pressure rises—which is when gas escapes from the airway. The design of peri-laryngeal supraglottic airway sealing devices thus far have sealing means that include a mechanism that involves a preset volume of air in sealing cuffs of laryngeal mask airways or in the case of the i-gel, a preset volume of soft and flexible solid (gel) cuff material. There are no dynamic self-energizing sealing perilaryngeal devices thus far designed and this limits the inflation pressures that can be used. It is the objective to have an airway sealing mechanism that will be responsive in an automatic way, to changes in airway dimensions and airway pressure, so as to minimize leak and to prevent ischemic or pressure damage to pharyngeal tissues which may occur with fixed constant volume sealing cushion devices—especially under circumstances of high sealing pressures.

It is a further object to achieve a higher seal pressure than has hitherto been possible with supraglottic airways. It is the intention that a self-energizing seal would provide the means to achieve this.

A further object is to design an airway that will be adaptable to different sizes of patient's throats so that one size will fit a greater range of patients. For pre-hospital care resuscitation circumstances, this is important, because a large range of devices is less portable.

A further object of the present invention is to provide a cushion-less means of sealing around the larynx so that variable dynamic sealing may occur in relation to the variation in airway pressure and, in turn, the accompanying anatomical shape variation, so as to avoid the potential damage caused by constant pressure over vulnerable areas within the throat that may occur with sealing mechanisms that occupy substantial space, said pressure that may be applied to the nerve near the tips of the patient's hyoid bones, namely the hypoglossal nerve, causing nerve dysfunction or discomfort or pain to the patient. Perilaryngeal sealing devices appear to run the risk of applying pressure indiscriminately to the vulnerable area near the tip of the hyoid bone and also that part of the recurrent laryngeal nerve that is located near the entrance to the oesophagus. It would therefore be beneficial to use a sealing mechanism that will achieve variable pressure applied to these vulnerable sealing sites, a pressure that is varied with airway pressure. In that way, the continuous seal pressure of inflatable cuffs or solid spongy cushion material over vulnerable areas is avoided, thereby minimizing the risk related to pressure damage.

A further object of the present invention is to provide for an anatomical shape that harmonizes with the anatomy in contrast with the attempt at providing sealing structures that oppose the perilaryngeal anatomy with a mirror image shape.

A further object of the present invention is to avoid using constant pressure sealing cushions over the vulnerable areas in the anterior aspect of the pharynx. So to achieve a more distal and posterior (behind) seal in a perilaryngeal sealing device that may help in avoiding pressure over the vulnerable hypoglossal nerve and nerve damage in the anterior (front) part of the perilaryngeal region.

It is a further object to manufacture the entire airway device from one single material including the attachment to active respiratory apparatus. The purpose is to achieve both improved safety in limiting disconnections that may happen if there is faulty adhesion of component parts that are made of different materials and to minimize expense in assembling different parts. This in turn makes for less expensive single use equipment—further enhancing safety in minimizing infection risk.

It is a further object to provide an airway that has a larger entrance for attachment to ventilation apparatus, so that easy passage of instruments is allowed, such as standard size tracheal tubes and armoured tubes, making the device more versatile for an increased number of applications.

Yet a further object is to provide an airway that uses all three known mechanisms for minimizing the risk of pulmonary aspiration of stomach contents and accumulated secretions. The three known mechanisms for preventing aspiration include (a) sealing the entrance into the oesophagus in the form of some obturator mechanism found in most supraglottic airways, (b) providing an escape route for oesophageal gases or liquids, thus far described only by means of drainage tubes, and (c) providing a means of trapping liquids within the device, described in its most effective way by means of the SLIPA airway. To maximize on effectiveness precludes the use of sealing cushion mechanisms that occupy substantial space/volume. Thus far devices have used one and sometimes two of these mechanisms but there are no devices that have used all three.

Another object is to retain airway sealing characteristics while at the same time, the provision of a means for passing a gastric tube via the mouth or via the nasal air passage. Thus far, there are no supraglottic airway devices that are designed to allow for the blind passage of a gastric tube via the nasal route.

Another object is the provision of a dead-space minimizing, bite-resistant device that can be placed or attached to the device, to make the device more suitable for spontaneous ventilation.

A final object is to minimise the risk of nosocomial chest infections caused by seepage of secretions past tracheal tube cuffs or pressure damage to structures in the laryngeal walls with the use of tracheal tubes, by designing a tracheal tube that will not have any folds that will allow for significant amounts of fluid seepage in the sealing surfaces or to control the size of the fold or folds so that its dimensions will minimize seepage of secretions to insignificant quantities and at the same time reduce perfusion pressure damage, secondary to high sealing pressures.

### SUMMARY OF THE INVENTION

According to one aspect of the present invention there is provided an airway device comprising a distal part and a proximal part in sealed communication;
said distal part defining a distal opening for attachment to a breathing apparatus, in sealed communication with a hollow stem; and
said proximal part comprising a hollow sealing chamber defining a proximal opening, said sealing chamber having means for sealing within and against the mucosal walls of a body cavity with a corresponding proximal opening, for communicating with the lungs;
wherein said sealing means of the sealing chamber includes: a longitudinal fold that allows for adaptation to varying cross-sectional dimensions of said body cavity; and said sealing chamber being made of an elastomeric substance and having walls that are thicker and relatively less flexible, and walls that are thinner and relatively more flexible, said thicker walls forming a framework that supports said thinner walls.

The thinner walls of the sealing chamber may be sufficiently thin to have elastic and flexible properties to permit expansion and contraction of the cross-sectional dimensions of the sealing camber in response to pressure within the chamber cavity as it rises and falls, so as to keep the walls of the sealing chamber in sealing contact with the walls of said body cavity.

The sealing chamber may be shaped and dimensioned to fit snugly within said body cavity, and the thicker walls may be thick enough to ensure that the shape of the sealing chamber is maintained during insertion of the airway device into the body cavity, to occupy the body cavity space and to hold the thinner walls suspended against the mucosal walls of the body cavity, to effect a self-energising seal.

The sealing chamber designed to fit within the trachea in the case of a tracheal tube version of the device or sealing chamber designed to fit within the pharynx in the case of a supraglottic airway version of the device based upon the same principle.

The body cavity may be pharynx and the longitudinal fold may comprise a gutter extending longitudinally along the length of the sealing chamber on a posterior (back, dorsal) aspect of the sealing chamber, such that the sealing chamber has a generally crescent-shaped cross-sectional profile with concavity in its posterior aspect.

The sealing chamber may be resiliently compressible to reduce its transverse dimensions, said compression being effected by side walls of said body cavity (the pharynx) compressing the sealing camber to decrease the radii of curvature of the anterior and posterior walls of the chamber and thereby bring the anterior and posterior walls of the chamber closer together.

The anterior wall of the sealing chamber may have a flexible thin free edge at the periphery of the proximal opening to enhance the self-energising seal and achieve variable pressure sealing.

The sealing chamber may form a blind pouch at its proximal end (leading end/toe) such that the flexibility of the walls of the sealing chamber allows said proximal end to expand with an increase in pressure inside the sealing pouch to enhance the self-energising seal to prevent air from escaping past the toe of the chamber and into the oesophagus.

The sealing chamber may include a flexible thin front wall in the distal (trailing) end of the sealing chamber, suspended by means of the thicker walls of the sealing chamber between its anterior wall and its attachment to the stem, said thin front wall being configured to seal the anterior of the chamber against the walls of said body cavity to prevent fluid under pressure in the airway device escaping “upwards” via the peri-laryngeal seal and into the mouth and then the atmosphere.

The sealing chamber may include a dynamic epiglottic elevator mechanism that is intrinsic within the design of the sealing chamber's shape, comprising the wall of the sealing chamber being thick near the stem (at the distal or stem-chamber junction aspect of the chamber) and the wall of the sealing chamber being thin and flexible with the free edge where sealing occurs at the base of the tongue. When in use, the thick wall at right angles to the gas flow path fits into the glosso-epiglottic fold between the base of tongue and the epiglottis and the thin-walled component is free to move in an anterior and distal direction thus lifting the epiglottis out of the way of the normal pathway for air flow during the inspiratory phase by means of the self-energizing sealing mechanism, in a position between the front surface and the lumen of the stem-chamber junction.

The shape of the sealing chamber may be convex at its anterior (towards the front) in a location that, in use, corresponds to the position of the hyoid bone in order to better match the shape of the hyoid bone, which is, likewise, convex shaped anteriorly.

The proximal opening in the front of the sealing chamber, surrounded by the thin walls of the sealing chamber may have transverse (perpendicular to the axis of the sealing chamber) dimensions that are large enough at a distal aspect of the proximal opening that corresponds to the position of the tips of the hyoid bone, in use, such that the free edge of the proximal opening is adjacent to the tips of the hyoid bone, thus ensuring maximum flexibility and minimal pressure against this vulnerable anatomical site.

The hollow nature and resilient compressibility of the sealing chamber may allow it to collapse in the anterior-posterior (front to back) direction, when inserting through the mouth and between the teeth of a patient—when there is a limited ability for the jaw to open.

The flexible, thin walls at the distal end of the sealing chamber may have a limited anterior surface or overhang dimension, supported by the thick walls that are transverse to an axis of the sealing chamber, to allow for outward bending and easy dislodgement of a trapped tongue, during placement (insertion) of the airway device.

The shape of the thicker stiffer walled back surface of the chamber may have a concave gutter running the (longitudinal) length of the chamber from trailing end to leading end, so that, when the device is in use, a space is created in the midline between the sealing posterior and lateral aspects of the sealing chamber and the posterior pharyngeal mucosa (P) behind, to allow for the preferential escape of any fluids (gases or liquids) that may enter the upper oesophagus, an escape via the space and mouth or the passage of a gastroscope (with comparative large bore). Also, the dorsal gutter provides for placement of a cuffed tracheal tube, which may be placed with tracheal cuff in the gutter and inflated in order to move the whole chamber anteriorly to ensure a good seal in very large pharynxes.

The resilient structure of the posterior wall gutter may allow for the insertion and guidance of a drainage tube that may be passed via nasal air passages or via the mouth into the oesophagus, said drainage tube being directed by said gutter into the pharyngeal space and thence into the entrance to the oesophagus and the stomach.

The cross-sectional shape of a distal part stem may be generally triangular with a rounded posterior apex, said triangular shape being formed by a thick anterior wall forming the flat base of the triangle, solid walls forming the sides of the triangle and thin back wall forming the rounded apex of the triangle, said cross sectional shape of the stem allowing for straight injection moulding of the stem and allowing the stem to bend preferentially in one direction with minimal collapse or risk of kinking of the lumen.

According to another aspect of the present invention there is provided an airway device comprising a distal part and a proximal part in sealed communication:
said distal part defining a distal opening for attachment to a breathing apparatus, in sealed communication with a hollow stem; and
said proximal part including means for forming a sealing chamber that is in flow communication with the stem and that has means for sealing against the mucosal walls of the pharynx of a patient and said proximal part defining a proximal opening, for communicating with the patient's lungs;
wherein said sealing means of the sealing chamber includes a blind pouch at its proximal end and the flexibility of walls of the sealing chamber allows said proximal end to expand with an increase in pressure inside the sealing pouch that allows for adaptation to varying cross-sectional dimensions of the proximal opening, said sealing chamber being made of an elastomeric substance and having walls that are thicker and relatively less flexible, and walls that are thinner and relatively more flexible, said thicker walls forming a framework that supports said thinner walls.

The term “chamber” is to be interpreted in this context as a substantially enclosed space and is to be interpreted broadly, so that its interpretation includes a space that is not necessarily completely hollow and that is surrounded by parts of the airway device, completely or in part—possibly with part of the patient's anatomy supplementing the enclosure of the space. The illustrated embodiments of the invention include a chamber formed by substantially enclosing a space with the proximal part of the airway device, but the present invention is not limited to such a structure.

The sealing chamber may include a flexible thin front wall in the distal end of the sealing chamber, suspended by means of the thicker walls of the sealing chamber between its anterior wall and its attachment to the stem, said thin front wall being configured to seal the anterior of the chamber against the walls of said body cavity.

The airway device may include at least one tubular passage through the stem and chamber from a distal end of the distal part to a proximal end of the chamber for the purpose of allowing liquids to escape or the possibility of passing a gastric tube into the oesophagus and stomach. The airway device may includes two of these tubular passages, each extending longitudinally along the stem and chamber with proximal openings at the proximal end of the chamber, said passages being spaced apart sufficiently to allow for the passage of a tracheal tube through the lumen of the stem and through the chamber between the passages.

The stem may be curved to conform to the shape of the generally right-angled bend from the oral cavity to the pharyngeal cavity of a patient, by means of a curved stiff insert that is receivable inside the lumen of the stem. The cross-sectional profile of the insert may be U-shaped so that one aspect of the lumen is flexible (the open aspect of the U is preferably posterior/behind) so that the facility of using curved optical stylets with the device combined with a bite block function, the two functions, normally being exclusive of one another. The chamber end of the insert has a shaped end with a lip protruding to prevent obstruction if kinking occurs as this point.

Alternatively, the device could be manufactured with an intrinsic curvature of the stem so as to make the insert redundant. The cross-section of the stem is rounded triangular cross-section, which lends itself to being bent easily with less collapsing of the lumen which normally occurs with bending, with some advantages of flexibility and space associated with the possibility of the device being used without the need for an insert.

The airway device may include an aspiration protection mechanism by virtue of the shape and configuration of its hollow chamber, which provides good capacity for storing and trapping secretions on the inside of the chamber for the purpose of minimizing the risk of accumulated or regurgitated secretions passing into the air passages.

The airway device may comprise a single component device made of one flexible and stretchable (elastic) material and the airway device may include an airway tube with attachment means at the distal opening for attaching to a breathing apparatus; said attachment means comprising a nominal 21-22 mm female connector site for friction fit attachment to the outside part of a standard 22 mm male tapered breathing attachment connector.

The large internal diameter channel in the distal female attachment end of nominal diameter of 21-22 mm, makes it possible to use standard size tracheal tubes and other rigid curved optical stylet instruments for the purpose of access to the bronchial tree via wide attachment and the stem with flexible posterior wall and proximal anterior opening in a pharyngeal sealing component.

The same self-energizing sealing mechanism can be employed in a tracheal tube design with the thin sealing walls of the chamber being held in position by an axial attachment along the length of the sealing walls, from a central thick walled tube for the purpose of holding the sealing walls against the mucosal walls in the trachea and, most importantly, to prevent the thin sealing walls from transverse folding during insertion.

The thin sealing walls may be free to expand and contract with changes in tracheal dimensions during the respiratory cycle by means of shaped longitudinal fold with a rolling seal.

The longitudinal sliding edge may comprise a narrow-angled wedge shape in cross-section that has a sliding edge with an extremely small radius of curvature (i.e. “sharp edge”) so as to minimize the effect of capillary fold leaks encountered in tracheal tubes.

The thin sealing walls may be free to expand and contract with changes in tracheal dimensions during the respiratory cycle resulting in variation in transverse diameter dimensions by means of one or more very thin-walled shaped longitudinal folds with a rolling seal and a slightly thicker, stiffer longitudinal sliding edge or edges if more than one longitudinal fold is applied.

The longitudinal sliding edge or edges comprises a narrow-angled wedge shape in cross-section that overlaps the expandable chamber, the sliding edge with an extremely small radius of curvature so that when it is sliding over the thin chamber wall which it overlaps, effectively reduces the size of the capillary fold leaks that may normally be encountered in tracheal tubes.

The proximal sealing chamber and shaft may be connected by a section that is narrowed in the lateral plane, so that the device may fit more comfortably between the cords with less distortion in an attempt to minimize long term distortion damage.

### BRIEF DESCRIPTION OF THE DRAWINGS

For a better understanding of the present invention and to show how it may be carried into effect, the invention will now be described by way of non-limiting example with reference to the accompanying drawings, in which:

FIG. 1 is a front three-dimensional view of a first embodiment of an airway device in accordance with the present invention, without a stem shaping insert;

FIG. 2 is a diagrammatic longitudinal (“coronal”—in medical terminology) sectional view of the airway device of FIG. 1;

FIG. 3 is a three-dimensional view of the airway device of FIG. 1 with a curved insert in the stem of the device;

FIG. 4 is a three-dimensional view of the stem shaping insert of the airway device of FIG. 3;

FIG. 5 is a longitudinal midline section, in situ, of the airway device of FIG. 1, being a straight version of the airway device that is curved naturally when the device is placed within the pharynx of a patient;

FIG. 6 is a view from a trailing end of the airway device of FIG. 1, in an inverted orientation;

FIG. 7 is a view from a leading end of the airway device of FIG. 1, in an upright orientation;

FIG. 8 is the same as in FIG. 2—repeated for ease of reference;

FIGS. 9 to 12 are cross-sectional views of the airway device of FIG. 8, taken at positions marked IX, X, XI and XII respectively, the positions of the sections being: at a toe of a chamber of the airway device in FIG. 9, at the middle of the chamber in FIG. 10, at the middle of a stem of the airway device in FIG. 11, and at an attachment end of the airway device in FIG. 12;

FIG. 13 is a longitudinal midline section, in situ, of the airway device of FIG. 1, to show by means of an arrow, the downward sealing mechanism of the toe of the device and the upward sealing mechanism at the base of the tongue;

FIGS. 14 and 15 are three-dimensional views of the airway device of FIG. 1, with constant sealing sites of the device shown with shading and with a thin walled sealing site pointed out by a human figure in FIG. 14;

FIG. 16 is a cross-sectional view of the chamber of the airway device of FIG. 1;

FIG. 17 is a cross-sectional view of the chamber of the airway device of FIG. 1, when forced into a smaller pharynx with side-wall pressure, the cross-section being shown with shading;

FIG. 18 is a combination of FIGS. 16 and 17—for easy comparison of change of the cross-section's shape;

FIG. 19 shows the cross section of FIG. 16, in combination with a similar cross-sectional view of the chamber of the airway device of FIG. 1, when squeezed through a narrowed gap between teeth, with accentuation of distortions of walls of the chamber;

FIG. 20 is a cross-sectional view of the chamber of the airway device of FIG. 1 (similar to FIG. 16) and shows the airway device in situ, in relation to the key human anatomy (convex front concave posterior hyoid bone) in the anterior (front) aspect, with sealing forces against the surrounding tissues shown by arrows, and showing the posterior pharyngeal wall;

FIG. 21 shows the interaction between the key human anatomy shown in FIG. 20, and prior art perilaryngeal supraglottic airway devices that are designed to mirror the anatomy (thus opposing the perilaryngeal sealing structures) thus differing in general as regards shape, with arrows showing sealing forces on the surrounding tissues;

FIG. 22 is a diagrammatic representation of the shape of a tracheal tube in accordance with the present invention, with stem and self energizing sealing chamber;

FIG. 23 is a three-dimensional view of the tracheal tube of FIG. 22;

FIG. 24 is a cross-section through the self-energizing chamber of the tracheal tube of FIG. 22; and

FIG. 25 is a longitudinal section through the tracheal tube of FIG. 22.

### KEY TO ANATOMICAL REFERENCES IN DRAWINGS

Ao Anterior oesophageal wall

B Base of tongue

Ep Epiglottis

Ge Glosso-epiglottic fold

BH Hyoid bone (FIGS. 20, 21)

Hp Hard palate

L Laryngeal inlet

M Mouth

N Nerve (Hypoglossal nerve) (FIGS. 20, 21)

Np Nasopharynx

O Oesophagus

P Posterior Pharyngeal Wall

Sp Soft palate

Te Teeth

T Tongue

Tr Trachea

U Uvula

### DETAILED DESCRIPTION OF THE DRAWINGS

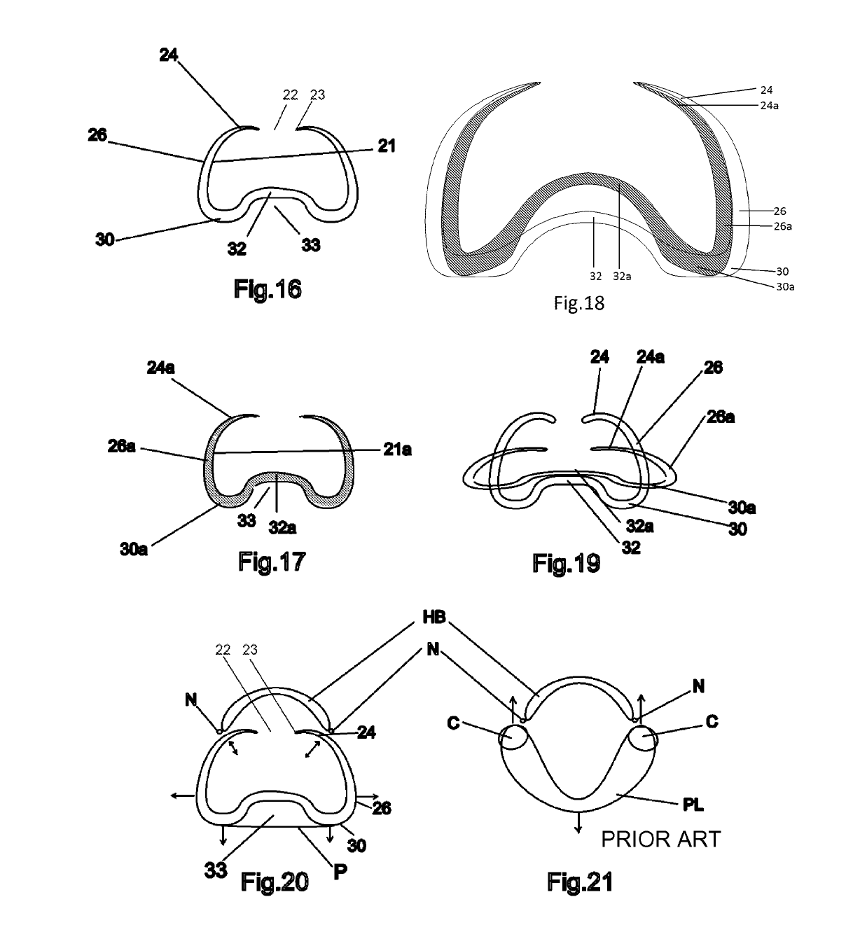

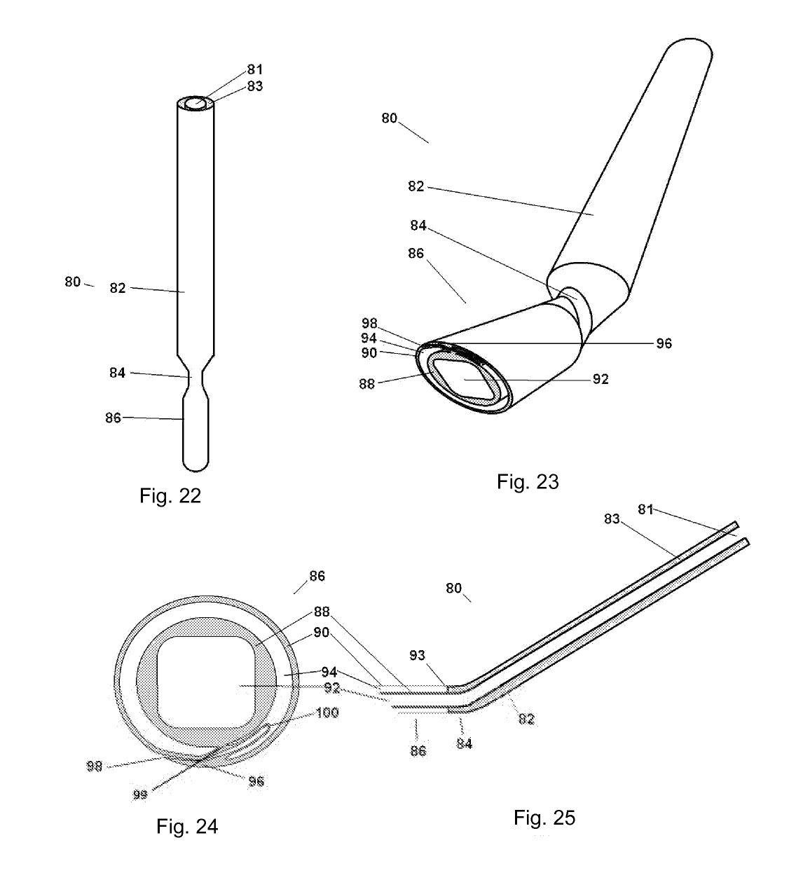

Referring to the drawings, an airway device in accordance with the present invention is generally indicated by reference numeral 10 in the case of the first supraglottic embodiment shown in FIGS. 1 to 21 and is indicated by reference numeral 80 in the case of the second supraglottic embodiment shown in FIGS. 22 to 25.

Referring to FIGS. 1 to 21, the airway device 10 comprises an airway tube divided into two parts with two open ends, proximal 20 and distal 12 parts each with respective proximal 22 and distal 14 openings. Proximal part 20 comprises a sealing chamber with means for sealing within and against the mucosal walls of a patient's body cavity with corresponding proximal opening, for communicating with the lungs, and the distal part 12 comprises a means for attachment to a breathing apparatus. The sealing chamber 20 has a longitudinal fold that allows for adaptation to varying cross-sectional dimensions of said body cavity, made of elastomeric substance with variable thickness walls, or more specifically, thick walls and thin walls, with the thick walls 30,26 providing a more rigid (less flexible) framework for supporting the more flexible and elastic thin walled parts 24,25 of the chamber in such a manner so as to ensure sealing contact of the thin walls of the chamber with the mucosal walls of the body cavity.

The thin walls 24,25 of the chamber 20 are sufficiently thin to allow for it to have elastic and flexible properties that permit expansion and contraction as the patient's airway cavity dimensions may expand and contract when airway pressure is raised or falls to a lower pressure such as ambient pressure—thereby achieving a self-energizing seal within the cavity.

Where the relevant body cavity is the pharynx and the longitudinal fold comprises a gutter 32 running the entire length of a sealing chamber 20 located in the dorsal or posterior aspect of the chamber, thus achieving a generally crescent shaped chamber in cross-section with concavity in the posterior or dorsal aspect.

The design of the chamber 20 is suited to fit within the pharyngeal cavity and the chamber's shape approximates a rounded wedge shape with proximal or leading end—which is the narrow end of wedge and is referred to as the ‘toe’ 28 and distal or trailing wider end to which a stem 16 of the distal part 12 is attached. The respective thin and thick walled parts of the chamber 20 with thin walled parts at the front and the front part of the sides 24 and the thick walled parts at the back 30 and the back part of the side walls 26 with generally flat front surface containing a large proximal or front opening 22. The front opening 22 is large enough to surround the larynx and corresponds with the laryngeal opening, with sufficient perimeter area around the opening on the thin walled side-front and front surface 24 to allow for dynamic or self-energizing sealing in the front around the larynx. The thicker walls 26,30 are thick enough to ensure that the chamber 20′s shape is maintained during insertion of the airway device 10 to occupy the pharyngeal space and to hold the thinner walled surfaces in the front (anterior) suspended against the pharyngeal walls to effect the self-energizing seal.

The self-energizing seal, which achieves variable pressure sealing, is made possible by flexibility of the front walls 24 that is enhanced by virtue of a free edge 23 around the periphery of the large front opening 22 in the chamber 20.

The self-energizing seal prevents air from escaping past a blind pouch formed at the leading end or toe 28 of the chamber 20 and into the oesophagus with the dimensions of the toe of the chamber expanding as the pressure rises.

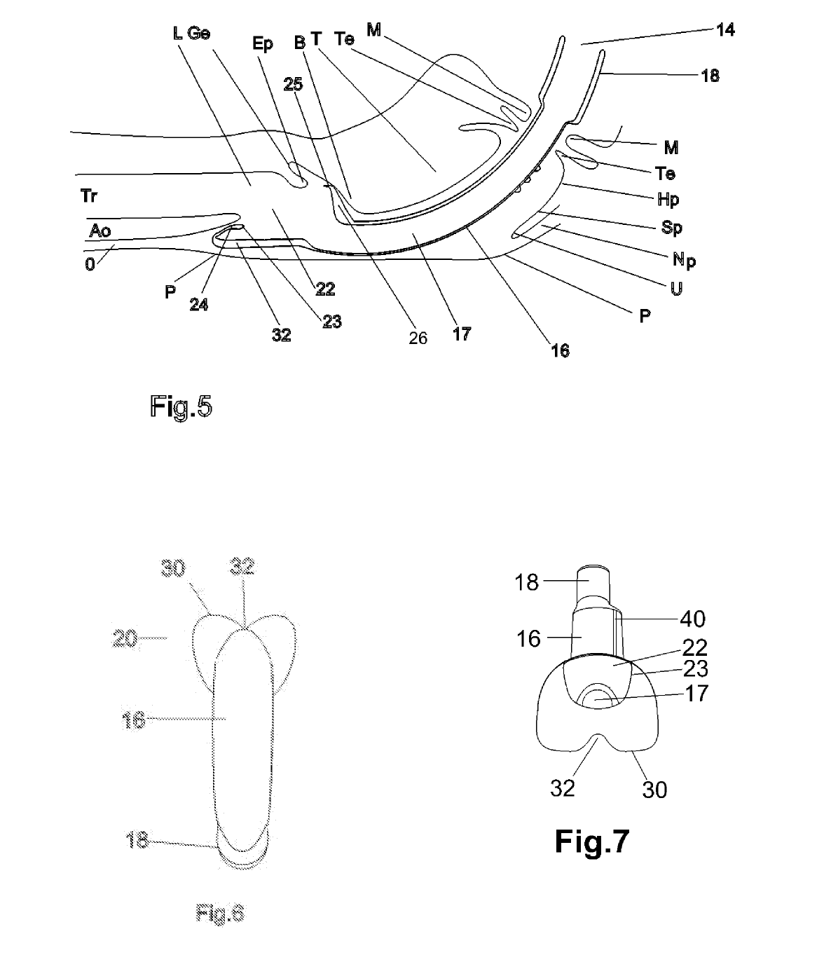

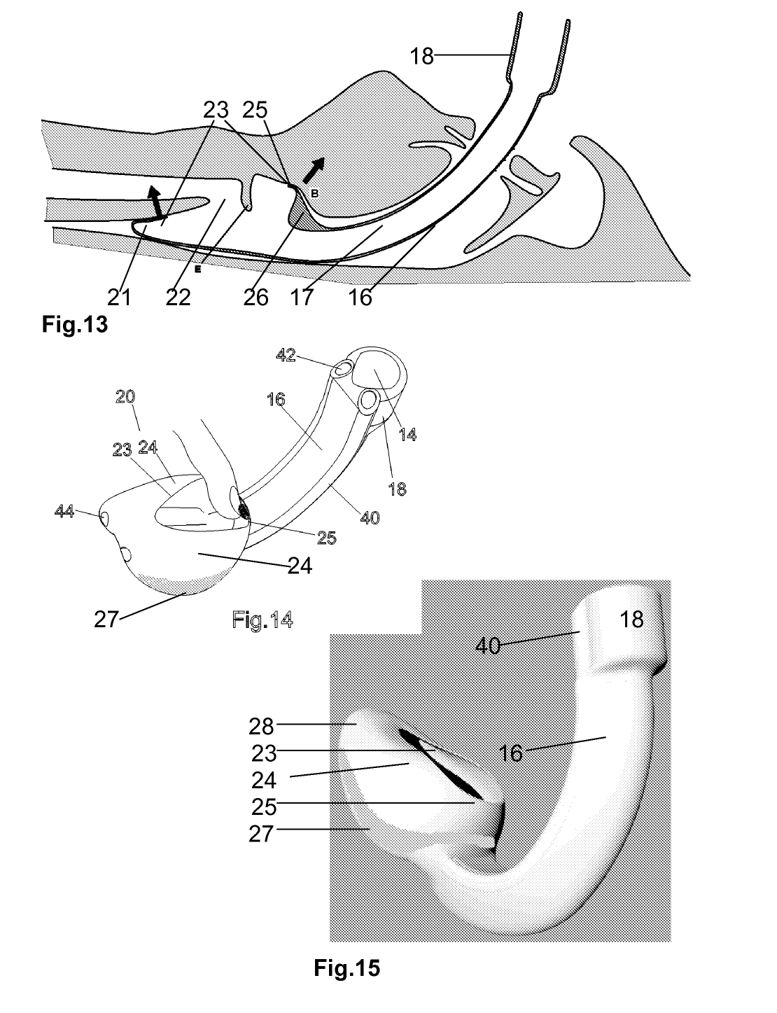

As can be seen in FIGS. 5 and 13, the sealing chamber 20 includes an oesophageal pouch “downward” seal formed by the front walls 24 that allows for adaptation to varying cross-sectional dimensions of inlet to the oesophagus.

The self-energizing sealing mechanism occurs by means of the thin movable front wall 24 and the thin movable wall 25 in the trailing end or distal aspect of the chamber 20, suspended by means of the thicker walls 26 between the front wall and the attachment of the stem 16, effectively sealing in the front to prevent gas under pressure in the airway device 10 escaping ‘upwards’ via the perilaryngeal seal and into the mouth and then the atmosphere. This could be described as a base-of-tongue pouch for the “upward” seal formed by the wall 25 (as seen in FIGS. 5 and 13) into the mouth.

The sealing chamber 20 forms a blind pouch at its proximal end (leading end/toe) (adjacent the front wall 24 as shown in FIG. 5, at the position of the left one of the two black arrows in FIG. 13), such that the flexibility of the walls of the sealing chamber allows said proximal end to expand with an increase in pressure inside the sealing pouch to enhance the self-energising seal to prevent air from escaping past the toe of the chamber and into the oesophagus.

The sealing chamber 20 may include a flexible thin front wall 25 in the distal (trailing) end of the sealing chamber, suspended by means of the thicker walls 26 of the sealing chamber 26 (as shown in FIGS. 5 and 13) between its anterior wall and its attachment to the stem, said thin front wall 25 being configured to seal the anterior of the chamber against the walls of the body cavity to prevent fluid under pressure in the airway device escaping “upwards” via the peri-laryngeal seal and into the mouth and then the atmosphere.

There is a dynamic epiglottic elevator mechanism intrinsic within the design (shape) of the chamber 20 comprising the distal or stem-chamber junction aspect of the chamber wall 26 being thickest near the junction of the stem 16 with chamber 20 and the very thin walled and flexible section 25 with free edge 23, where sealing occurs at the base of the tongue. When in use, the thick wall 26 at the junction of the stem 16 to the chamber 20, is oriented transversely to the longitudinal gas flow path through the chamber 20 and fits into the glosso-epiglottic fold between the base of tongue and the epiglottis, whereas the thin-walled part 25 is free to move in an anterior and distal direction, thus lifting the epiglottis out of the way of the normal pathway for air flow during the inspiratory phase by means of the self-energizing sealing mechanism, in a position between the front surface 24 and a lumen 17 of the stem-chamber junction.

The chamber 20 size is more adaptable to various sizes of patient anatomy by virtue of the rounded shape of the chamber, where the front 24 and back 30,32 walls are concave towards the back (i.e. convex towards the front), so that, in cross-section, the airway device 10 may appear to be rounded or crescent-shaped in cross-section, with the radius of curvature of the back wall 30 smaller than the combined general radius of curvature of the front wall 24 and front aspect of the side wall. The chamber 20′s structure shaped as a rounded, generally crescent structure in cross-section has a collapsing characteristic in that compressing forces from the side walls of a patient's pharynx may accentuate the curvature of the crescent, thus decreasing both front and back radii of curvature and bringing the front and back surfaces closer together and making the overall transverse dimensions of the chamber smaller. Conversely, a rise in airway pressure may exert pressure on the insides 21 of the walls of the chamber 20, which will tend to increase radius of curvature of front and back walls—thus enlarging the sealing dimensions of the chamber.

The cross-sectional shape of the chamber 20 is convex anteriorly (towards the front) that in use would correspond at the level of the hyoid bone (HB) (as shown in FIG. 20) in order to match the shape of the hyoid bone better, which is also convex shaped, anteriorly.

The large opening 22 in the front of the chamber 20 in the thin walled area 24 may be wide enough (dimension transverse to the chamber's axis) in the distal aspect of the opening that corresponds to the larynx at the same level as the tips of the hyoid bone, so that the free edge 23 of the laryngeal opening 22 is near to these tips of the hyoid bone—thus ensuring flexibility and minimising pressure against this vulnerable anatomical site.

The flexible hollow chamber 20 structure allows for effective collapsing in the front to back direction by virtue of it being flexible, hollow and crescent-shaped, when inserting through the mouth and between the teeth, when there is a limited ability for the jaw to open (as shown in FIG. 19).

The limited front surface or overhang dimension of the very thin walled flexible sealing part 25 at the distal end of the chamber 20 supported by a thick walled part 26 generally transverse to the chamber axis, allows for outward bending (as shown by the arrow in FIG. 13) and easy dislodgement of a trapped tongue during insertion.

The shape of the stiffer thicker wall 30 of the chamber 20 has a concave gutter 32 running the (longitudinal) length of the chamber from trailing end to leading end, so that, when the device 10 is in use, a space 33 is created in the midline between the sealing posterior and lateral aspects 32,30,26 of the sealing chamber and the posterior pharyngeal mucosa P behind (as shown in FIG. 20), to allow for the preferential escape of any fluids (gases or liquids) that may enter the upper oesophagus, an escape via the space 33 and mouth or the passage of a gastroscope (with comparative large bore).

The resilient structure of the posterior wall gutter 32 allows for the insertion and guidance of a drainage tube into the oesophagus and the stomach that may be passed via nasal air passages or via the mouth and may be directed by the gutter 32 into the pharyngeal space 33 between outside walls 32,30 of the chamber 20 and the posterior pharyngeal wall P (as shown in FIG. 20) and thence into the entrance to the oesophagus.

The cross-sectional shape of the distal part stem 16 is generally triangular with rounded apex posteriorly with its thick anterior wall 19a forming the flat base of the triangle, its solid triangular side walls 19c and thin back wall 19b forming an apex (as sown in FIG. 11) to allow for a straight injection moulded airway tube to bend preferentially in one direction with minimal collapse or risk of kinking of the lumen 17.

There may be one or more tubular passages 40 through the stem 18,16 and chamber 20 from the attachment 42 to the leading edge 44 of the chamber or toe 28 for the purpose of allowing liquids to escape or the possibility of passing a gastric tube into the oesophagus and stomach.

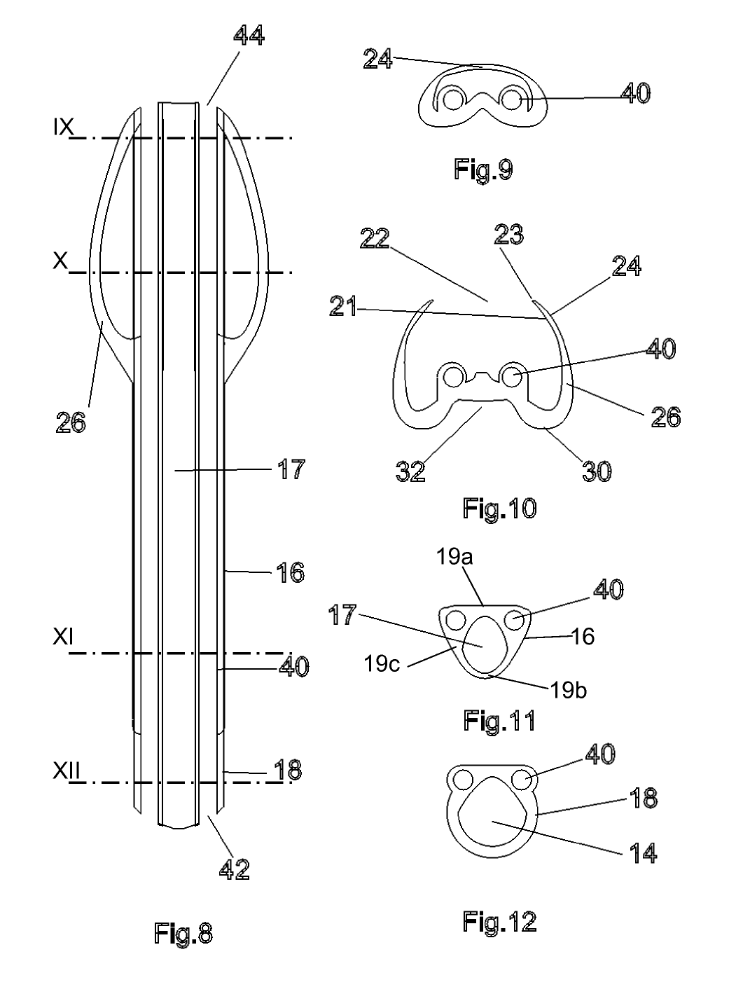

Two parallel channels 40 (as shown in FIGS. 3 and 8) are provided along the length of the stem 12 and chamber 20 with proximal openings into the leading end 28,44 of the chamber 20, with sufficient distance between the parallel channels to allow for the passage of a tracheal tube through the stem lumen 17 and chamber and between the parallel channels 40.

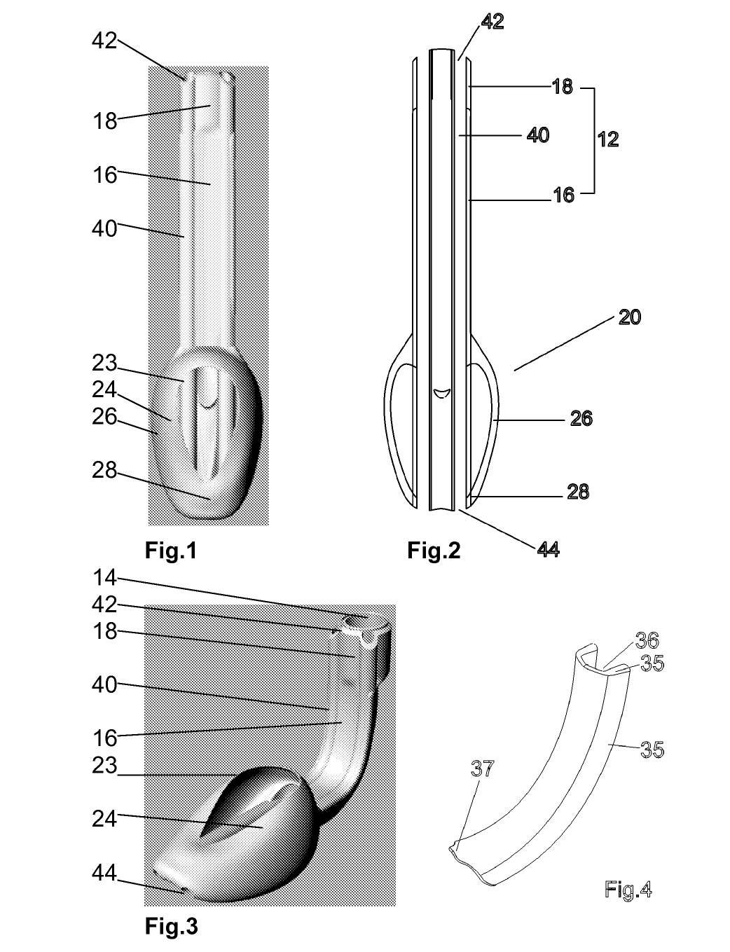

The shape of the stem 16 is curved (as shown in FIG. 3) to conform to the shape of the generally right-angled pharyngeal cavity by means of a curved stiff plastic insert 35 (shown in FIG. 4) placed within the airway lumen 17 of the stem.

It is possible for the device 10 to be used without the curved plastic insert 35 (as shown in FIGS. 1, 2 and 8). The rounded triangular cross-section of the stem 16 (shown in FIG. 11) lends itself to being bent easily with less collapsing of the lumen 17 than normally occurs with bending, with some advantages of flexibility and space associated with the possibility of the device 10 being used without the need for an insert 35.

The shape of the curved stiff plastic insert 35 in cross-section is U-shaped (or “channel”-shaped) so that one aspect of the airway lumen 17 is flexible (the open aspect 36 of the U is preferably posterior/behind). This allows the use of curved optical stylets with the device 10, in combination with the insert 35, which serves as a bite block. Prior to the present invention, the use of bite blocks and curved optical stylets have normally been exclusive of each other. The chamber end of the insert has a shaped end with a lip 37 in FIG. 4 protruding to prevent obstruction if kinking occurs as this point.

The hollow design of the chamber 20 serves as an effective aspiration protection mechanism, by providing good capacity for storing and trapping secretions on the inside of the chamber, for the purpose of minimizing the risk of accumulated or regurgitated secretions passing into the air passages.

Apart from the optional insert 35, this device 10 may be manufactured as a single component device made of one flexible and stretchable (elastic) material comprising an airway tube with attachment means to a breathing apparatus. The distal opening 14 at the distal attachment 42 in a distal part 18 of the stem 12 comprises a nominal 21-22 mm female connector site for friction fit attachment to the outside part of the standard 22 mm male tapered breathing attachment connector.

The large internal diameter of the distal opening 14 in the distal female attachment 42 of nominal diameter of 21-22 mm, makes it possible to use standard size tracheal tubes and other rigid curved optical stylet instruments for the purpose of access to the bronchial tree via opening 14 and the stem 16 and lumen 17 and anterior proximal opening 22 in a pharyngeal sealing component or chamber 20.

One distinctive feature of the sealing mechanism of the present invention is the longitudinal folding mechanism that provides a constant pressure seal away from the vulnerable anterior region immediately in the perilaryngeal region. So far, all supraglottic (SG) perilaryngeal sealing airways except for the Baska airway, with its dynamic “anterior concertina fold”, have had fixed constant pressure sealing cushions that may be classified as sealing in the perilaryngeal (e.g. laryngeal mask and i-gel airways) or base of tongue regions (e.g. Combitube, Copa airway, Laryngeal Tube, SLIPA airway). However, the airway device 10 has a fixed constant pressure seal somewhere between these two regions, the shaded area 27 shown in FIGS. 14 and 15 and a different self-energizing dynamic sealing mechanism in the immediate perilaryngeal region over the front surface and anterior aspect of side walls 24. The constant pressure perilaryngeal cushion in other perilaryngeal sealers (PL) such as laryngeal mask or i-gel airways (shown as C in FIG. 21) comprising air inflated cushions or the gel cushion of the i-gel airway, are both located immediately surrounding the glottic or laryngeal opening, where they seal. The sealing mechanisms in the present invention are twofold being both constant pressure cushion sealing site 27 and dynamic or self-energizing by virtue of the tendency for airway pressure within the device to unfold the crescent shaped chamber 20 and effect an enhanced seal over the front surfaces of more flexible walls 24 and 25. The dynamic sealing site in this invention is in the immediate peri-laryngeal region and indeed the whole pharynx when airway pressure unfolds the device 10. Constant pressure sealing site 27, however, may also be referred to as in a perilaryngeal region although not in the immediate perilaryngeal area but further away from certain vulnerable anatomical structures in the front (anterior) but instead is located in the posterior region of the side walls 26 and widest dimensions of the back walls 30.

In order to inhibit seepage of secretions that could cause nosocomial chest infections, a tracheal tube design with a self-energizing seal is now described with reference to FIGS. 22 to 25, which shows an airway tracheal tube device 80 made of one flexible material comprising an airway tube divided into two parts 82 and 86 and with two open ends 81 and 92.

One of the parts of the device 80 is a chamber 86 which has thin sealing walls 90 that may be held in position by axial attachment 99 along the length of the sealing walls 90 from a central thick walled tube 88 for the purpose of holding the sealing walls against the mucosal walls in the trachea and, most importantly, to prevent the thin sealing walls from transverse folding during insertion.

The thin sealing walls 90 are free to expand and contract with changes in tracheal dimensions during the respiratory cycle by means of a shaped longitudinal fold with a rolling seal 100.

A longitudinal sliding edge 96 of the device 80 comprises a narrow-angled wedge shape in cross-section that overlaps the expandable chamber 90. The edge 98 of the sliding edge 96 has an extremely small radius of curvature (i.e.“sharp edge”) so that when it is sliding over the thin chamber wall 90 with which it overlaps, it effectively reduces the size of the capillary fold and minimises capillary fold leaks that may normally be encountered in tracheal tubes.

In FIGS. 24 to 27, the following numbers in earlier drawings may be substituted by the equivalent functional components with the numbers in brackets following: 14 (81), 22 (92), 12 (82), 20 (86, 90), 24 & 25 (90), 30 & 26 (88), the general cross-sectional crescent shape including 24, 26 and 30 about the concave longitudinal groove 32 (100). The sealing chamber 86 is designed to fit within the trachea in the case of the tracheal tube version 80 of the device and the sealing chamber 20 is designed to fit within the pharynx in the case of a supraglottic airway version 10 of the device, based upon the same principle.

The thin sealing walls 90 are free to expand and contract with changes in tracheal dimensions during the respiratory cycle resulting in variation in transverse diameter dimensions by means of one or more very thin-walled shaped longitudinal folds 100 with a rolling seal and a slightly thicker stiffer longitudinal sliding edge 96 or, in preference, two or more edges if more than one longitudinal fold is employed in the design.

In FIGS. 23 and 24, the proximal sealing chamber 86 and shaft 82 may be connected by a section 84 that is narrowed in the lateral plane, so that the device 80 may fit more comfortably between the cords with less distortion in an attempt to minimize long term distortion damage.

1-29. (canceled)

30. An airway device for sealing a patient's pharynx without penetrating into the larynx, said airway device comprising a distal part and a proximal part in sealed communication;
said distal part defining a distal opening for attachment to a breathing apparatus, in sealed communication with a hollow stem; and
said proximal part comprising a sealing member with means for sealing within and against the mucosal walls of the patient's pharynx, and said proximal part defining a proximal opening that is in communication with the hollow stem for communicating with the patient's lungs,
wherein said sealing means of the sealing member includes a longitudinal fold extending longitudinally along the length of the sealing member on a posterior aspect of the sealing member, such that the sealing member has a generally crescent-shaped cross-sectional profile with concavity in its posterior aspect.

31. An airway device according to claim 30, wherein the sealing member is a hollow flexible sealing chamber that is shaped to conform to the anatomy of the pharynx.

32. An airway device according to claim 31, wherein the sealing chamber is resiliently compressible to reduce its transverse dimensions, said compression being effected by side walls of the patient's pharynx compressing the sealing camber to decrease radii of curvature of anterior and posterior walls of the sealing chamber and thereby bring the anterior and posterior walls of the sealing chamber closer together.

33. An airway device according to claim 32, wherein the anterior wall of the sealing chamber has a flexible thin free edge at a periphery of the proximal opening.

34. An airway device according claim 31, wherein the sealing chamber forms a blind pouch at a proximal end of the sealing chamber, such that the flexibility of the walls of the sealing chamber allows said proximal end to expand with an increase in pressure inside the sealing pouch.

35. An airway device according to claim 31, wherein the sealing chamber includes a flexible thin front wall in a distal end of the sealing chamber, said flexible thin front wall being suspended by means of thicker walls of the sealing chamber between an anterior wall of the sealing chamber and an attachment of the sealing chamber to the stem, said thin front wall being configured to seal the anterior of the sealing chamber against the walls of the patient's pharynx.

36. An airway device according to claim 31, wherein the sealing chamber includes a dynamic epiglottic elevator mechanism comprising a wall of the sealing chamber being thick near an attachment of the sealing chamber to the stem and a wall of the sealing chamber being thin and flexible with a free edge where sealing occurs at the base of the tongue, the position of said thin wall of the sealing chamber corresponding to the position of the patient's epiglottis.

37. An airway device according to claim 30, wherein the shape of the sealing member is convex at its anterior in a location that, in use, corresponds to the position of the patient's hyoid bone.

38. An airway device according to claim 30, wherein the proximal opening has transverse dimensions that are large enough at a distal aspect of the proximal opening that corresponds to the position of the tips of the hyoid bone, in use, such that a free edge of the sealing member around the periphery of the proximal opening is adjacent to the tips of the patient's hyoid bone.

39. An airway device according to claim 31, wherein the sealing chamber is resiliently compressible and the hollow nature and resilient compressibility of the sealing chamber allows it to collapse in the anterior-posterior direction, when inserting through the mouth and between the teeth of the patient.

40. An airway device according to claim 31, wherein flexible, thin walls at a distal end of the sealing chamber have limited anterior surface dimensions, supported by the thick walls that are transverse to an axis of the sealing chamber, to allow for outward bending and easy dislodgement of the patient's tongue if it becomes trapped during placement of the airway device.

41. An airway device according to claim 31, wherein the sealing chamber has a resilient, relatively thick posterior wall that is concave in shape to define the longitudinal fold, such that, in use, a pharyngeal space is created in the longitudinal fold between the posterior wall and the posterior pharyngeal mucosa of the patient, said pharyngeal space allowing for the escape of fluids and/or the passage of an instrument.

42. An airway device according to claim 41, wherein the resilient structure of the posterior wall longitudinal fold allows for the insertion and guidance of a drainage tube into the patient's oesophagus, said drainage tube being directed by said longitudinal fold into the patient's pharyngeal space and thence into the entrance to the patient's oesophagus.

43. An airway device according to claim 30, wherein the cross-sectional shape of a distal part the stem is generally triangular with a rounded posterior apex, said triangular shape being formed by a thick anterior wall forming a flat base of the triangle, solid walls forming sides of the triangle and a thin back wall forming the rounded posterior apex of the triangle, said cross sectional shape of the stem allowing for straight injection moulding of the stem and allowing the stem to bend with minimal collapse or risk of kinking.

44. An airway device according to claim 31, wherein said sealing chamber is made of an elastomeric substance and has walls that are thicker and relatively less flexible, and walls that are thinner and relatively more flexible, said thicker walls forming a framework that supports said thinner walls.

45. An airway device according to claim 44, wherein the thinner walls of the sealing chamber are sufficiently thin to have elastic and flexible properties to permit expansion and contraction of the cross-sectional dimensions of the sealing camber in response to variations of pressure within the cavity of the sealing chamber, so as to keep the walls of the sealing chamber in sealing contact with the walls of the patient's pharynx.

46. An airway device according to claim 44, wherein the sealing chamber is shaped and dimensioned to fit snugly within the patient's pharynx, and said thicker walls being thick enough to ensure that the shape of the sealing chamber is maintained during insertion of the airway device into the patient's pharynx, to occupy the patient's pharynx and to hold the thinner walls suspended against the mucosal walls of the patient's pharynx, to effect a self-energising seal.

47. An airway device according to claim 31, wherein said airway device includes at least one tubular passage through the stem and chamber from a distal end of the distal part to a proximal end of the chamber.

48. An airway device according to claim 47, wherein said airway device includes two of said tubular passages, each extending longitudinally along the stem and chamber with proximal openings at the proximal end of the chamber, said passages being spaced apart sufficiently to allow for the passage of a tracheal tube through a lumen of the stem and through the chamber between said passages.

49. An airway device according to claim 30, wherein the stem is curved to conform to the shape of the bend from the oral cavity to the pharyngeal cavity of the patient, by means of a curved stiff insert that is receivable inside a lumen of the stem.

50. An airway device according to claim 49, wherein the cross-sectional profile of said insert is U-shaped so that one aspect of the lumen is flexible.

51. An airway device according to claim 30, wherein said airway device comprises a single component device made of one flexible and elastic material, and said airway device including attachment means at the distal opening for attaching to a breathing apparatus; said attachment means comprising a nominal 21-22 mm female connector site for friction fit attachment to the outside of a standard 22 mm male tapered breathing attachment connector.

52. An airway device comprising a distal part and a proximal part in sealed communication:
said distal part defining a distal opening for attachment to a breathing apparatus, in sealed communication with a hollow stem; and
said proximal part including means for forming a sealing chamber that is in flow communication with the hollow stem and that has means for sealing against the mucosal walls of the pharynx of a patient, and said proximal part defining a proximal opening, for communicating with the patient's lungs;
wherein said sealing means of the sealing chamber includes a blind pouch at a proximal end of the chamber and the flexibility of walls of the sealing chamber allowing said proximal end to expand with an increase in pressure inside the sealing pouch that allows for adaptation to varying cross-sectional dimensions of the proximal opening, said sealing chamber being made of an elastomeric substance and having walls that are thicker and relatively less flexible, and walls that are thinner and relatively more flexible, said thicker walls forming a framework for supporting said thinner walls.

53. An airway device according to claim 52, wherein the sealing chamber includes a flexible thin front wall in the distal end of the sealing chamber, suspended by means of the thicker walls of the sealing chamber between its anterior wall and its attachment to the stem, said thin front wall being configured to seal the anterior of the chamber against the walls of the patient's pharynx.

54. An airway device according to claim 52, wherein said airway device includes at least one tubular passage through the stem and chamber from a distal end of the distal part to a proximal end of the chamber.

55. An airway device according to claim 54, wherein said airway device includes two of said tubular passages, each extending longitudinally along the stem and chamber with proximal openings at the proximal end of the chamber, said passages being spaced apart sufficiently to allow for the passage of a tracheal tube through a lumen of the stem and through the chamber between said passages.

56. An airway device according to claim 52, wherein the stem is curved to conform to the shape of the bend from the oral cavity to the pharyngeal cavity of the patient, by means of a curved stiff insert that is receivable inside a lumen of the stem.

57. An airway device according to claim 56, wherein the cross-sectional profile of said insert is U-shaped so that one aspect of the lumen is flexible.

58. An airway device according to claim 52, wherein said airway device comprises a single component device made of one flexible and elastic material, and said airway device including attachment means at the distal opening for attaching to a breathing apparatus; said attachment means comprising a nominal 21-22 mm female connector site for friction fit attachment to the outside part of a standard 22 mm male tapered breathing attachment connector.
